(19) United States
(12) Patent Application Publication
(10) Pub. No.: US 2019/0314648 A1
(43) Pub. Date: Oct. 17, 2019

(54) METHOD OF SKIN CARE

(71) Applicant: THE BOOTS COMPANY PLC, Nottingham (GB)

(72) Inventors: Aneshkumar Dineshchandra Sitaram, Birstall, Leicester (GB); Jake Thomas Hicks, Beeston, Nottingham (GB); Paul James Tomlinson, Derby (GB); Michael David Bell, Alfreton, Derbyshire (GB)

(21) Appl. No.: 16/314,256

(22) PCT Filed: Jun. 27, 2017
(86) PCT No.: PCT/EP2017/025182

§ 371 Date: Dec. 28, 2018

(30) Foreign Application Priority Data
Jun. 30, 2016 (GB) 1611413.4

(51) Int. Cl. A61Q 19/08; A61K 8/44; A61K 8/60; A61K 8/67; A61K 8/9789; A61K 8/49

(52) U.S. Cl. CPC A61Q 19/08; A61K 8/447; A61K 8/602; A61K 2800/78; A61K 8/9789; A61K 8/498; A61K 8/676

(57) ABSTRACT

The disclosed technology relates to a method of decreasing or preventing signs of ageing of décolleté skin and optionally the face and/or neck skin comprising topically applying to the skin a composition comprising a cosmetically acceptable medium, a vitamin C, or derivative thereof, and compound having two or more hydroxyl groups, wherein the compound has a molar mass of at least 150 g/mol and increases transglutaminase K expression in epidermal keratinocytes.

### TECHNICAL FIELD

The disclosed technology relates to a method of decreasing or preventing signs of ageing of décolleté skin and optionally the face and/or neck skin comprising topically applying to the skin a composition comprising a cosmetically acceptable medium, a vitamin C, or derivative thereof, and compound having two or more hydroxyl groups, wherein the compound has a molar mass of at least 150 g/mol and increases transglutaminase K expression in epidermal keratinocytes.

### BACKGROUND OF THE INVENTION

Ageing is a multifactorial phenomenon. The ageing phenomenon may be due to one or more of genetic predisposition (known as chronological or intrinsic ageing) and one's physiological reaction to environmental stresses (sometimes referred to as actinic or extrinsic ageing). Actinic ageing appears skin specific and is defined as the effect of the external environment on the skin's biological response. The skin response to actinic ageing, which may be caused by sun and pollution exposure, as well as smoking, is typically associated with a lack of normal hydration, apparition of telangiectasia (spider veins), sagging of the skin, and/or reduced firmness of the skin. With sagging or reduced firmness the appearance of fine lines and wrinkles occurs. The sagging or reduced skin firmness may be explained by the fact that the elastic fibres of the dermal extracellular matrix, forming the support and conferring elasticity and strength to the skin are destroyed and become rare with age.

For example, one of the pathways to influence ageing involves the epidermal enzyme transglutaminase, a key driver of terminal keratinocyte differentiation and the development of the cornified envelope. The cornified envelope allows the epidermis to maintain a healthy and strong skin barrier, and its production. Transglutaminase, and its production is influenced by epidermal calcium concentration. When switched to high calcium concentrations (the calcium switch), cells in the stratum basale are believed to begin differentiating by producing involucrin, a component of the cornified envelope, and the enzyme, transglutaminase-K (TGK). This is believed to then be responsible for the crosslinking of involucrin and other substrates into the insoluble cornified envelope and form intercellular contacts important for the differentiation process.

In addition, it is known that although skin covers body, its thickness varies depending on the amount of wear and tear that body parts experience. On the face, neck, and décolleté skin differs from the rest of the body because skin is thinner than on most parts of the body. However, these three skin regions of the body frequently experience most exposure to environmental stresses, and other signs of ageing. As a result signs of ageing of skin on face, neck and décolleté age are obvious to see, and because of biological difference between different skin regions of the body they age at different rates.

When comparing face, neck, and décolleté skin there are differences. For example the décolleté is generally believed to have the thinnest skin on the body, and has the fewest oil glands. Face and neck skin similarly differ as neck skin is thinner than facial skin, and it has fewer oil glands than facial skin.

Thus the factors influencing ageing vary depending on the location and type of skin.

In order to find a solution to reducing the signs of ageing, a number of studies have been undertaken looking at the form of some skin care or cosmetic compositions containing ingredients such as ascorbic acid, or derivatives thereof. The compositions disclosed relate to treating signs of ageing of facial skin. The applications include:

U.S. Pat. No. 2,400,171 (Ruskin, published 14 May 1946), U.S. Pat. No. 6,146,664 (Siddiqui, published 14 Nov. 2000), U.S. Pat. No. 8,022,090 (Choi et al., published 20 Sep. 2011), US2007196310 (Mary Kay, published 21 Feb. 2007), US 2014/0155633 (Lin-Chao et al, published 5 Jun. 2014), U.S. Pat. No. 7,741,496 (Wei-Chuan et al., published 22 Jun. 2010) and US 2011/02811943 (Pei Miu et al., published 17 Nov. 2011), U.S. Pat. No. 6,110,476 (Nguyen et al., published 29 Aug. 2000), US 2008/0253982 (Shibayama, published 16 Oct. 2008), EP2722043 A1 (Lin et al., 23 Apr. 2014), U.S. Pat. No. 6,162,419 (Perricone et al., published 19 Dec. 2000), U.S. Pat. No. 6,087,393 (Mathur, published 7 Jul. 2000), U.S. Pat. No. 8,053,469 (8 Nov. 2011), U.S. Pat. No. 5,736,567 (Cantin et al., published 7 Apr. 1998), U.S. Pat. No. 5,140,043 (Darr et al., published 18 Aug. 1992), U.S. Pat. No. 6,010,706 (Candau et al., published 4 Jan. 2000), U.S. Pat. No. 6,036,963 (Weinkauf et al, published 14 Mar. 2000), US 2008/0287533 (Gupta, published 20 Nov. 2008), GB patent applications GB1519184.4 and GB1519200.8 (filed 31 Oct. 2015 and published under GB2543818 and GB2543822).

### SUMMARY OF THE INVENTION

The disclosed technology may be used to decrease or prevent at least one of the following forming wrinkles or fine lines, skin sagging, or hyperpigmentation (such as solar lentigines), or increasing skin firmness or skin laxity. The skin includes décolleté, and optionally may include one or both of the face, and neck.

In one embodiment the disclosed technology relates to a composition having efficacy to increase the synthesis of transglutaminase-K (TGK); and that is believed to be beneficial for the appearance of healthy skin.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of and “consisting of,” where “consisting of excludes any element or step not specified and “consisting essentially of permits the inclusion of additional un-recited elements or steps that do not materially affect the basic, essential and novel characteristics of the composition, method or use under consideration.

Unless otherwise indicated treat rates are on a weight basis relative to the total composition disclosed herein.

Unless otherwise indicated various ingredients disclosed herein may be individual compounds, or in a mixture of compounds.

In one embodiment the disclosed technology relates to a method of decreasing or preventing the signs of ageing (such as decreasing or preventing at least one of the following: forming wrinkle or fine lines, skin sagging, or increasing skin firmness) of décolleté skin and optionally the face and/or neck skin comprising topically applying to the skin a composition comprising a cosmetically acceptable medium, a vitamin C, or derivative thereof, and a compound having two or more hydroxyl groups, wherein the compound has a molar mass of at least 150 g/mol.

In one embodiment the disclosed technology relates to a method of decreasing or preventing signs of ageing of décolleté skin and optionally the face and/or neck skin comprising topically applying to the skin a composition comprising a cosmetically acceptable medium, a vitamin C, or derivative thereof, and compound having two or more hydroxyl groups, wherein the compound has a molar mass of at least 150 g/mol and increases transglutaminase K expression in epidermal keratinocytes.

In one embodiment the disclosed technology relate to the use of a composition disclosed herein to decrease or prevent at least one of the following: forming wrinkles or fine lines, skin sagging, or to increase skin firmness or skin laxity of décolleté skin and optionally the face and/or neck skin, wherein the composition comprises a cosmetically acceptable medium, a vitamin C, or derivative thereof, and a compound having two or more hydroxyl groups, wherein the compound has a molar mass of at least 150 g/mol.

In one embodiment the method/use disclosed herein is for the décolleté, and neck skin.

In one embodiment the method/use disclosed herein is for the décolleté, and face skin.

In one embodiment the method/use disclosed herein is for the décolleté, face and neck skin.

In one embodiment the method/use disclosed herein is capable of increasing/promoting the activity of transglutaminase by topically applying to décolleté skin and optionally the face and/or neck skin the composition disclosed herein.

In one embodiment the disclosed technology relates to a composition is in the form of a cream, lotion or serum.

The use and method disclosed herein are known to the skilled person as not encompassing therapeutic or medical treatment i.e., the disclosed use or method relate to a non-therapeutic use or method.

Skin may be a mammalian skin such as human skin.

### DETAILED DESCRIPTION OF THE INVENTION

The disclosed technology provides a composition, methods and uses as disclosed above.

### Vitamin C or Derivative Thereof

The composition comprises vitamin C, or derivative thereof and may include ascorbic acid, O-substituted ascorbic acid (or derivative thereof), sodium ascorbyl phosphate, ascorbyl glycoside, L-ascorbic acid, ascorbyl palmitate, retinyl ascorbate, tetrahexyldecyl ascorbate, or magnesium ascorbyl phosphate.

The O-substituted ascorbic acid or derivative thereof may be an O-alk(en)yl ascorbic acid or derivative thereof.

As used herein “alk(en)yl” is intended to mean alkyl or alkenyl (for example alkyl).

The alk(en)yl may be acyclic or cyclic, for example acyclic. The acyclic group may be linear or branched, for example linear.

In one embodiment the O-substituted ascorbic acid or derivative thereof may be an O-alkyl ascorbic acid, or derivative thereof.

The O-substituted ascorbic acid, or derivative thereof is known in the art, and described in EP Patent application EP2722043 A1, and US 2014/0155633 (both Lin et al., Applicant Corum).

In one embodiment the O-substituted ascorbic acid, or derivative thereof may be represented by the formula:

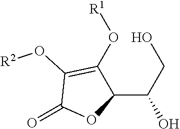

wherein R¹ and R² groups may independently be H, C1-20 alkyl, C3-20 cycloalkyl, C1-20 alkoxy, C2-20 acyl, C6-20 aryl, C1-20 heterocyclic aromatic, C1-20 heterocyclic non-aromatic, or C3-20 cycloalkenyl.

The O-substituted ascorbic acid or derivative thereof may have a substituted group that may be hydrocarbon in nature i.e., composed on carbon and hydrogen. In one embodiment R¹ and R² groups may independently be H, C1-20 alkyl, C3-20 cycloalkyl, C6-20 aryl, or C3-20 cycloalkenyl.

In one embodiment the O-substituted ascorbic acid may be 3-alkyl ascorbic acid, or mixtures thereof. For example the alkyl group may be a C1-20, or C1-10, or C2-8, or C2-4.

In one embodiment the 0-substituted ascorbic acid may be 3-ethyl ascorbic acid.

The vitamin C or derivative thereof may be present at 0.001 to 5 wt %, or 0.01 to 3 wt %, or 0.1 to 2 wt % of the composition.

### Compound

It has surprisingly been found that a compound having two or more hydroxyl groups, a molar mass of at least 150 g/mol and that increases TGK expression in epidermal keratinocytes, when combined with an O-substituted ascorbic acid or derivative thereof as described above, is particularly effective in improving the firmness, reducing the photodamage and reducing the uneven skin tone of the décolleté after topical application. Determining whether a compound having two or more hydroxyl groups and a molar mass of at least 150 g/mol is capable of increasing TGK expression in epidermal keratinocytes can be directly and positively verified by a person skilled in the art through, for example, a standard anti-TGK ELISA assay as described in Study 1. ELISA assays are so readily carried out that such analysis would not be considered to be a form of undue experimentation.

The composition disclosed herein comprises a compound having two or more hydroxyl groups, wherein the compound has a molar mass of at least 150 g/mol, or at least 160 g/mol (herein referred to as “the compound”. The molar mas may be 150 to 2000 g /mol, or 160 to 1000 g/mol, or 160 to 700 g/mol, or 200 to 500 g/mol.

The compound may have 2 to 150, or 2 to 30, or 2 to 10, or 2 to 3, or 2 hydroxyl groups.

The compound may be ionic, or non-ionic.

The compound may be aromatic, or non-aromatic.

In one embodiment, the compound may be a hydroxymethionine or one of its salts and/or derivatives. A hydroxymethionine salt may include a metal salt, such as calcium, or magnesium salt of 2-hydroxymethionine.

The metal salt may be a calcium salt of 2-hydroxymethionine (for example with a molar mass of about 338.4 g/mol). The calcium salt comprises two hydroxyl groups due to the presence of one hydroxyl per anion of the salt (since there are two moles of 2-hydroxymethionine per one mole of calcium cations).

The hydroxymethionine salt may be described in more detail in EP2209460. EP2209460 describes a composition comprising a mixture of homotaurine and a hydroxymethionine, or one of its salts and/or derivatives (for example the calcium salt).

In one embodiment the compound may be an aromatic compound such a polyphenol.

In one embodiment the compound may be derived from an extract of a plant. The extract of a plant may be a polyphenol. In different embodiments the extract may be chosen from one or more of:

(i) Centella asiatica (commercially available as Centevita™), or

(ii) Cistus incanus (commercially available as Retorcyl™), or

(iii) Polygonum bistorta (commercially available as Perlaura™), or

(iv) Myrothamnus flabellifolia (commercially available as Myramaze™)

In a different embodiment the compound may be an isoflavone such as genistein (commercially available as Lipobelle Soyaglycone from Mibelle). Genistein may have chemical name 5,7-dihydroxy-3-(4-hydroxyphenyl)chromen-4-one. The isoflavone may be described in more detail in US2008/0299092, US2012/0195948, and US2014/0072619.

In one embodiment the compound is non-aromatic.

The compound may be glyceryl glucoside. Glyceryl glucoside has six hydroxyl groups.

The compound may be present in the composition in an amount ranging from 0.0001 wt % to 10 wt %, or 0.0003 wt % to 5 wt %, or 0.003 wt % to 3.5 wt % of the composition. In one embodiment the compound may be present at 0.1 wt % to 4 wt %, or 1 wt % to 3.5 wt % of the composition.

Preferably the compound is selected from the list consisting of a salt and/or derivative of hydroxymethionine (preferably a calcium salt of hydroxymethionine), genestein, a Centella asiatica extract, a Cistus incanus extract, a Polygonum bistorta extract and a Myrothamnus flabellifolia extract.

### Other Ingredients

The composition disclosed herein may optionally further comprise other ingredients. The other ingredients include Hibiscus, a peptide, a matrix metalloproteinase inhibitor (MMPi), a whitening agent, a skin conditioning agent, a salicylic acid compound, a sunscreen agent, preservatives thickeners, viscosity modifying agents, and/or gelling agents sequestering agents, wax, diluents, carriers, propellants perfumes, or pH adjusting agents.

In one embodiment the composition disclosed herein further comprises one or more of Hibiscus, a peptide, an MMPi and a whitening agent.

The Hibiscus may be Hibiscus sabdariffa, Hibiscus rosa sinensis or Hibiscus Abelmoschus. All three Hibiscus plants are known to form extracts used in cosmetic compositions. The Hibiscus may be in the form of an extract.

Peptides are defined as compounds comprising an uninterrupted sequence of amino acids. For example the peptides are of natural origin. A dipeptide comprises an uninterrupted sequence of two amino acids. Amino acids, as employed herein, include and encompass all of the naturally occurring amino acids, either in D or L configuration. Amino acids are commonly indicated with reference to the conventional three letter code and the sequence is read from left to right. The composition of the disclosed technology may comprise a dipeptide chosen from acetyl dipeptide 1 cetyl ester, acetyl dipeptide 3 aminohexanoate, azelaoyl bisdipeptide 10, coumaroyl dipeptide 3, dicetyl dipeptide 9, dipeptide diamino butyroyl benzylamide diacetate, dipeptide 1, dipeptide 10, dipeptide 11, dipeptide 12, dipeptide 15, dipeptide 16, dipeptide 17, dipeptide 18, dipeptide 19, dipeptide 2, dipeptide 20, dipeptide 3, dipeptide 4, dipeptide 5, dipeptide 6, dipeptide 7, dipeptide 8, dipeptide 8 HCL, dipeptide 9, hexanoyl dipeptide 3 norleucine acetate, methyl undecylenoyl dipeptide 16, nicotinoyl dipeptide 22, nicotinoyl dipeptide 23, nicotinoyl dipeptide 24, nicotinoyl dipeptide 26, oleoyl dipeptide 15, palmitoyl dipeptide 10, palmitoyl dipeptide 13, palmitoyl dipeptide17, palmitoyl dipeptide 5 diaminobutyroyl hydroxythreonine, palmitoyl dipeptide 5 diaminohydroxybutyrate, palmitoyl dipeptide 7 and mixtures thereof.

In one embodiment the composition of the disclosed technology may comprise a tripeptide, or mixtures thereof. The tripeptide may be naturally occurring or of synthetic origin. Suitable tripeptide compounds include tripeptide 1, 2, 3, 4, 5, 6, 7, 8, 9, 10,11, 12, 13, 14, 15, 16, 17, 18, 19, 20, 21, 22, 23, 24, 25, 26, 27, 28 ,29, 30, 31, 32, 33, 34, 35, 36 ,37, 38, 39, 40, 41, 42, 43, 44, 45, 46, derivatives thereof, and mixtures thereof. The tripeptide comprise one or more His-based tripeptides.

The compositions of the disclosed technology may further comprise a tetrapeptide. The tetrapeptide may be one or more rigin-based tetrapeptides, one or more ALAMCAT-tetrapeptides or mixtures thereof. The tetrapeptide may be naturally occurring or of synthetic origin. Suitable tetrapeptides for use in the present composition include those chosen from tetrapeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 34, 35, derivatives thereof and mixtures thereof.

Rigin-based tetrapeptides of the disclosed technology may be based on the structure Gly-Gln-Pro-Arg (Rigin) and include its analogues and derivatives thereof. Rigin is a typical tetrapeptide.

The compositions of the disclosed technology may further comprise a pentapeptide, derivatives of thereof, and mixtures thereof. As used herein, “pentapeptide” refers to both the naturally occurring pentapeptide and synthesized pentapeptide. Also useful herein are naturally occurring and commercially available compositions that comprise pentapeptides. Suitable pentapeptides are those chosen from pentapeptide 1, 4, 5, 6, 7, 8, 9, 10, 11, 12, 13, 14, 15, 16, 17, 18, 19, 21, 22, 23, 24, 25, 26, 28, 29, 30, 31, 33, 34, 35, 36, 38, 39, derivatives thereof and mixtures thereof.

The peptide when present in the composition disclosed herein may be present at 0 or 0.01% to 20%, or 0.05% to 15%, or 0.05 to 10 wt % of the composition.

### Matrix Metalloproteinase Inhibitor (MMPi)

The term “matrix metalloproteinase inhibitor” relates to all molecule and/or plant or bacterial extracts having an inhibitory activity on at least one of the matrix metalloproteinases expressed, synthetized, or activated by or in the skin. The family of MMPis is formed of several well-defined groups on the basis of their resemblance regarding structure and substrate specificity (Woessner J. F., Faseb Journal, vol. 5, 1991, 2145).

The MMPi may be present at a level of from 0 or 0.01% to 10%, or 0.1% to 5% or 0.25% to 2.5%, or 0.5% to 1% by weight of the composition.

### Whitening Agent

In one embodiment the composition disclosed herein further comprises a whitening/lightening agent.

When the composition further comprises the whitening/lightening agent it may be present at 0 or 0.001 to 10 wt %, or 0.01 to 5 wt %, or 0.1 to 2 wt %, or 0.2 to 1 wt % of the composition. For example the whitening/lightening agent may be present at 0 or 0.001% to 3 wt %, or 0.01 to 2 wt%, or 0.05 to 1 wt %, or 0.1% to 0.5 wt % of the composition.

The whitening/lightening may include at least one of the following ingredients: Emblica, Mulberry leaf extract, mangostin, Sophora, a flavonoid, hydroxyphenoxy propionic acid and dimethylmethoxy chromanol.

In one embodiment the whitening/lightening agent may be a mixture of ingredients chosen from: Emblica and Sophora, optionally in the presence of Mulberry leaf extract. For example the Mulberry leaf extract may be present.

The Emblica may be Emblica officinalis, for example comprising over 40% by weight (for example 50-80 wt %) of Emblicanin A. Emblicanin B, Pedunculagin and Punigluconin, and not more than about 1% by weight of flavonoids. The Emblica may be phyllanthus Emblica.

The Sophora may be an extract of a small tree, and shrub in the pea family Fabaceae.

The Emblica may be phyllanthus Emblica and Sophora is derived from Sophora Angustifolia Root Extract.

The flavonoid species is believed to have antioxidant performance, and be an antioxidant plant polyphenolic agent. By the term antioxidant plant polyphenolic agent we mean a plant extract, or derivative thereof, comprising flavonoid species, including flavones, flavonols, flavanones, flavanols anthrocyanidins and isoflavonoids; phenolic acid species; stilbenes; lignans and mixtures thereof, which provide an antioxidant benefit. Antioxidant benefit is measured using the total antioxidant capacity (TAC) assay described herein. Plants provide a rich source of polyphenolic agents, and are therefore an efficient source of said antioxidants. Similar actives may be prepared synthetically and as such are analogues of said plant polyphenolic agents.

Antioxidant polyphenolic agents (different from the compound having two or more hydroxyl groups, wherein the compound has a molar mass of at least 150 g/mol) may include extracts from plants chosen from Mulberry (e.g. Morus alba), Ginseng (e.g. Panax ginseng), Raspberry, Oregano (e.g. Origanum vulgare), Green tea (e.g. green leaves of Camellia sinensis), White tea (e.g. Camellia sinensis), Blueberry extract (e.g. Vaccinium cyanococcus), French maritime pine bark (e.g. Pinus pinaster, sold under the trade name Pycnogenol), Rosemary (e.g. Rosmarinus officialis), Grape, including grape seed (e.g. Vitis vinifera), Fennel (e.g. Foeniculi fructus), Caragana sinica, Marjoram (e.g. Origanum majorana), Crocus (e.g. Crocus sativus), Apple (e.g. Malus domestica), Coffee, Green coffee, Cherry (e.g. Prunus avium), Snow algae (e.g. Chlamydomonas nivalis), Emblica (e.g. Phyllanthus emblica), Gingko (e.g. Gingko biloba), Moringa (e.g. Moringa oleilera), Ginger (e.g. Zingiberaceae), Magnolia (e.g. Magnolioideae virginiana), French saffron, Edelweiss (e.g. Leontopodium alpinium), White lotus (e.g. Nymphaea alba), Turmeric root, Marshmallow (e.g. Althaea officianlis), Burdock (e.g. Arctium lappa), Bilberry (e.g. Vaccinium myrtillus), Cranberry (e.g. Vaccinium oxycoccus), Pomegranate nectar (e.g. Punica granatum), Sage (e.g. Salvia officinalis), Thyme (e.g. Thymus vulgaris), Sunflower (e.g. Helianthus annuus), wild carrot (e.g. Daucus carota), Hop (e.g. Humulus lupulus), Witch Hazel (e.g. Hamamelis), Oak (e.g. Quercus), Camellia (e.g. Theacea), Red clover (e.g. Tritolium pratense), Flax (e.g. Linium usitatissimum), lemon (e.g. Citrus limon), birch (e.g. Betula), cornflower, (e.g. Centaurea cyanus), geranium, polygonum, soy (e.g. Glycine max), and mixtures thereof.

In one embodiment the antioxidant polyphenolic agent may be an extract from a plant chosen from mulberry, ginseng, grape, oregano, grape, sage, sunflower, maritime pine bark, rosemary, marjoram, crocus, french saffron, wild carrot, hop, coffee, green coffee, witch hazel, oak, camellia, red clover, flax, ginger, magnolia, edelweiss, burdock and mixtures thereof.

Active polyphenolic species sourced from the above list of plants include those chosen from apigenin, luteolin, quercetin, kaempferol, naringenin, hesperetin, catechin, gallocatechin, cyaniding, pelargonidin, daidzein, caffeic acid, chlorogenic acid, romsmarinic acid, gallic acid, resveratrol, ferulic acid, epigallocatechin gallate, piceatannol, secoisolariciresinol, isotaxiresinol, Miyabenol c, Luteolin and mixtures thereof.

The amounts of antioxidant plant polyphenolic agents used in the present invention are expressed as dry weights of the extract, as understood by a man skilled in the art. The antioxidant plant polyphenolic agent (plant extract) may be present at 0.005 to 10 wt %, or 0.01 to 7 wt %, or 0.01 to 5 wt % of the composition.

When present the chromane may be chosen from: methyl, di-, tri- and tetra- C1-C6 alkyl, C1-C6 alkoxy chromanol; pentamethyl chromanol, methyl, di, tri and tetra C1-C6 alkyl, C1-C6 alkoxy chromanyl C14-C20 ester and mixtures thereof.

The chromane may be chosen dimethyl methoxy chromanol, tetramethyl methoxy chromanol, pentamethyl chromanol, dimethyl methoxy chomanyl palmitate, dialkyl methoxy chomanyl myristate, dimethyl methoxy chromanyl stearate, dimethyl methoxy chomanyl oleate, dimethyl methoxy chomanyl linoleate and mixtures thereof.

In one embodiment the chromane may be dimethyl methoxy chromanol (commercially available under the trade name Lipochroman 6 as sold by Lipotec).

### Skin Conditioning Agent

The composition disclosed herein may optionally comprise a skin conditioning agent. The skin conditioning agents may be chosen from humectants, emollients, moisturisers, or mixtures thereof. Where present, the skin conditioning agent may be present from 0.01 to 20 wt %, or 0.1 to 10 wt %, or 0.5 to 7 wt % of the composition.

The skin conditioning agents may be chosen from guanidine, urea, glycolic acid and glycolate salts, salicylic acid, lactic acid and lactate salts, aloe vera, shea butter, polyhydroxy alcohols, such as sorbitol, mannitol, xylitol, erythritol, glycerol, hexanetriol, butanitriol, (di) propylene glycol, butylene glycol, hexylene glycol, polyethylene glycol, sugars (e.g. fructose, glucose, xylose, honey, mannose, xylose), gluconodeltalactone, and starches and their derivatives, pyrrolidone, carboxylic acid, hyaluronic acid and salts thereof, lactamide monoethanolamine, acetamide monoethanolamine, panthenol, allantoin and mixtures thereof.

For example the skin conditioning agent may be chosen from glycerine, arabinoglactan, butylene glycol, hyaluronic acid, shea butter, propylene glycol, ethylhexyl glycerine, hyaluronate and mixtures thereof

### Salicylic Acid Compound

The compositions disclosed herein may optionally comprise a salicylic acid compound, its esters, its salts, or combinations thereof. In one embodiment of the composition disclosed herein comprises a salicylic acid compound at 0.0001 to 25 wt %, or 0.001 to 15 wt %, or 0.01 to 10 wt %, or 0.1 to 5 wt %, and or 0.01 to 2 wt% of the composition, of salicylic acid. In one embodiment the salicylic acid compound may be salicylic acid.

### Sunscreen

The composition disclosed herein may optionally comprise a sunscreen component. The sunscreen may comprise organic or inorganic sun filters or a combination of the two. Suitable inorganic sunfilters include those chosen from microfine titanium dioxide, and microfine zinc oxide, and mixtures thereof.

Suitable organic sunscreens include those chosen from: a) p-aminobenzoic acids, their esters and derivatives (for example, 2-ethylhexyl p-dimethylaminobenzoate), b) methoxycinnamate esters (for example, 2-ethylhexyl p-methoxycinnamate, 2-ethoxyethyl p-methoxycinnamate or a, p-di-(p-methoxycinnamoyl)-a′-(2ethylhexanoyl)-glycerin, c) benzophenones (for example oxybenzone), d) dibenzoylmethanes such as 4-(tert-butyl)-4′-methoxydibenzoylmethane, e) 2-phenylbenzimidazole-5 sulphonic acid and its salts, f) alkyl-ss, ss-diphenylacrylates for example alkyl a-cyano-ss, ss-diphenylacrylates such as octocrylene, g) triazines such as 2,4,6-trianilino-(p-carbo-2-ethyl-hexyl-1-oxi)-1,3,5 triazine, h) camphor derivatives such as methylbenzylidene camphor and i) mixtures thereof. Other sunscreen ingredients include those chosen from homosalate, Ethylhexyl salicylate, Diethylhexylbutamido triazone, Bis-ethylhexyloxyphenol methoxyphenyl triazine, Diethylamino hydroxybenzoyl hexyl benzoate, Butyl methoxydibenzoylmethane, Methylene bis-benzotriazoyl tetramethylbutylphenol, Polysilicone-15 and mixtures thereof. A sunscreening agent may be present from 0 to 10 wt %, or 0.1 to 10 wt % of the composition.

### Other Optional Ingredients

The compositions disclosed herein may also optionally comprise one or more of the following optional ingredients. Preservatives may be added to the composition such as benzoic acid, sodium benzoate, sorbic acid, potassium sorbate, 2-bromo2-nitropropane-1,3-diol (bronopol, which is available commercially under the trade name Myacide®, benzyl alcohol, diazolidinyl urea, imidazolidinyl urea, methyl paraben, phenoxyethanol, ethyl paraben, propyl paraben, sodium methyl paraben, sodium dehydroacetate, polyhexamethylenebiguanide hydrochloride, isothiazolone and sodium propyl paraben and mixtures thereof, suitably in an amount of from 0.01 to 10 wt % of the composition.

Sequestering agents may be added to the composition, such as ethylenediamine tetraacetic acid and salts thereof, for example in an amount of from 0.005 to 0.5 wt % of the composition.

The composition may also include waxes such as cocoa butter suitably in an amount of from 0.1 to10 wt % of the composition.

The composition may also comprise suitable, cosmetically acceptable diluents, carriers and/or propellants such as dimethyl ether. The composition may also include pearlising agents such as stearic monoethanolamide and/or mica, suitably in an amount of from 0.01 to 10 wt % of the composition.

Perfumes may be added suitably in an amount of from 0.01 to 2 wt % of the composition, as may water soluble dyes such as tartrazine, suitably in an amount of from a trace amount such as 1×10⁻⁵ to 0.1 wt % of the composition.

The composition may also include pH adjusting agents such as sodium hydroxide, amino methyl propanol, triethanolamine, suitably in an amount of from 0.01 to 10 wt % of the composition. The composition may be buffered by means well known in the art, for example by use of buffer systems comprising succinic acid, citric acid, lactic acid, and acceptable salts thereof, phosphoric acid, mono-or disodium phosphate and sodium carbonate. Suitably, the composition may have a pH between 3 and 10, between 4 and 8, or between 4.5 and 6.5.

In one embodiment the composition of the disclosed technology does not include MMP compounds that comprise one hydroxyaryl or polyhydroxyaryl compound, or cyclic compounds having a cyclic group based upon a compound comprising a pyran, a lactam, or a piperidine constituent.

### Cosmetically Acceptable Medium

The cosmetically acceptable medium may be water, alcohol, or an oil. In one embodiment the cosmetically acceptable medium may include water and/or an oil.

The composition disclosed herein may be in the form of a gel or an emulsion.

As used herein reference to gel is used in the ordinary sense defined by IUPAC and is intended to include a non-fluid colloidal network or polymer network that is expanded throughout its whole volume by a fluid. The fluid may for instance be water or alcohol. In one embodiment the fluid is water.

When the composition disclosed herein is in the form of an emulsion, the emulsion disclosed herein may be a water-in-oil, oil-in-water, or water-in-silicone composition, for example an oil-in-water, or water-in-silicone composition, often oil-in-water.

The emulsion may comprise an oil phase and have an aqueous phase content of 30 to 85 wt %, or 40 to 80 wt %, or 50 to 75 wt % of the composition.

The emulsion may comprise an oil phase having 15 to 70 wt %, or 20 to 50 wt %, or 25 to 50 wt % of the composition.

The emulsion may be an oil-in-water composition comprising 15 to 70 wt % of an oil phase; and 30 to 85 wt % of an aqueous phase, or comprising 25 to 50 wt % of an oil phase; and 50 to 75 wt % of an aqueous phase.

The emulsion may be in the form of a water-in-silicone emulsion, and the water phase may be present at 30 to 85 wt % of an aqueous phase; and silicone present at 15 to 70 wt % of a silicone phase.

The emulsion may be in the form of a water-in-silicone emulsion, and the water phase may be present at 60 to 75 wt % of an aqueous phase; and silicone present at 25 to 40 wt % of a silicone phase.

If the composition disclosed herein is in the form of a water-in-silicone composition the oil phase may be provided by any suitable silicate, dimethiconols, silicone elastomer and mixtures thereof (for example a silicone elastomer).

For example the silicone oil phase may be formed from an organopolysiloxane. The organopolysiloxane may be chosen from one or more of a polyalkylsiloxane, alkyl substituted dimethicone, cyclomethicone, trimethylsiloxysilicate. dimethiconol, polyalkylaryl siloxane, and mixtures thereof. The polyalkylsiloxane may be for example a cyclomethicone, or dimethicone, for example a dimethicone.

A water-in-silicone composition disclosed herein may include an emulsifying crosslinked organopolysiloxane elastomer, a non-emulsifying crosslinked organopolysiloxane elastomer, or a mixture thereof. The term “non-emulsifying,” as used herein, defines crosslinked organopolysiloxane elastomers from which polyoxyalkylene units are absent. The elastomers may include dimethyl polysiloxanes crosslinked by Si—H sites on a molecularly spherical MQ resin. Emulsifying crosslinked organopolysiloxane elastomers include the crosslinked polymers described in U.S. Pat. Nos. 5,412,004; 5,837,793; and 5,811,487. The emulsifying elastomer comprised of dimethicone copolyol crosspolymer (and) dimethicone is commercially available from Shin Etsu under the trade name KSG-21.

The non-emulsifying elastomers may include dimethicone crosspolymers. Such dimethicone crosspolymers are supplied by a variety of suppliers including Dow Corning (EL9240). Other dimethicones crosspolymer are available from General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the composition disclosed herein and processes for making them are further described in U.S. Pat. Nos. 4,970,252; 5,760,116; and 5,654,362. Commercially available elastomers typical for use herein are Dow Coming's 9040 silicone elastomer blend, Shin Etsu's KSG-21, and mixtures thereof.

An oil-in-water or water-in-oil emulsion may comprise an organic oil. The organic oil may be volatile or non-volatile. The organic oil may include a diluent, a solvent, a polyolefin polymer, or an ester oil.

The term “ester oil” means an oil that is liquid at room temperature (25° C.) comprising at least one ester functional group. The ester oil used herein is chosen, for example, from monoesters.

The ester oil may, for example, be chosen from the monoesters of formula R¹COOR² wherein R¹ may be selected from linear and branched hydrocarbon-based chains comprising from 4 to 30, or 6 to 24, or 7 to 20 carbon atoms carbon atoms, and R² may be chosen from branched hydrocarbon-based chains comprising from 3 to 40 carbon atoms, such as from 10 to 30 carbon atoms and further such as from 16 to 26 carbon atoms.

Examples of the ester oils that may be mentioned include isodecyl neopentanoate; isocetyl octanoate; isononyl isononanoate, isodecyl isononanoate, tridecyl isononanoate; hexyl laurate, 2-hexyldecyl laurate; isopropyl myristate, isocetyl myristate, isotridecyl myristate, 2-octyldodecyl myristate; isopropyl palmitate, 2-ethylhexyl palmitate, isooctyl palmitate, isocetyl palmitate, isodecyl palmitate, isostearyl palmitate, 2-octyldecyl palmitate; isopropyl isostearate, 2-octyldodecyl stearate, isostearyl isostearate, and 2-octyldodecyl erucate.

The ester oil may be present in the emulsion disclosed herein in an amount ranging, for example, from 0 to 20 wt %, or 0.1 to 15 wt %, or 1 to 10 wt % of the composition.

### EXAMPLES

### Study 1

Comparative Example 1 (CE1): is a composition comprising 0.03 wt % of diospyros.

Comparative Example 2 (CE2): is a composition comprising 0.03 wt % of a commercial product MEIRITAGE™ (mixture of Astragalus Membranaceus Root Extract (and) Atractyloides Macrocephala Root Extract (and) Bupleurum Falcatum Root Extract).

Comparative Example 3 (CE3): is a composition comprising 0.001 wt % of an Iris florentina root extract (commercially available as Iris Iso OP™)

Example 1 (EX1): is a composition comprising 0.00005 wt % of genistein from a Liposome soy isoflavone (commercially available from Mibelle).

Example 2 (EX2): is a composition comprising 0.001 wt % of a calcium salt of hydroxymethionine (commercially available as Essenskin™ ceramide from Sederma; and is a mixture comprising homotaurine and calcium salt of hydroxymethionine).

Example 3 (EX3): is a composition comprising 0.003 wt % of a Centella asiatica extract (commercially available as Centevita™)

Example 4 (EX4): is a composition comprising 0.0003 wt % of a Cistus incanus extract (commercially available as Retorcyl™)

Example 5 (EX5): is a composition comprising 0.03 wt % of a Polygonum bistorta extract (commercially available as Perlaura™)

Example 6 (EX6): is a composition comprising 0.001 wt % of a Myrothamnus flabellifolia extract (commercially available as Myramaze™)

Example 7 (EX7): is a composition comprising 0.001 wt % of a Glyceryl Glucoside.

### Testing

Each example is evaluated by an in vitro procedure. The in vitro procedure uses a cell culture derived from human epidermal keratinocytes that have been cultured at 37° C. in 5% carbon dioxide. The culture medium is Keratinocyte-SFM supplemented with epidermal growth factor (0.25 ng/ml), pituitary extract (25 μg/ml, and gentamycin (25 μg/ml). The culture assay medium is Keratinocyte-SFM supplemented with gentamycin (25 μg/ml).

The cell culture is then analysed for TGK expression. The cells are seeded in 96-well plates and cultured for 24 hours in the culture medium. The medium is then replaced with an assay medium containing EX1 to EX7 and the cells incubated for 72 hours. All experimental conditions are performed in N=3.

At the end of incubation, the assay medium is discarded and the cells are rinsed, fixed and premeabilized. The cells are then labelled using a primary antibody. The primary antibody are then revealed using corresponding appropriate fluorescent secondary antibodies, and the cell nuclei are coloured using Hoescht solution 33258 (bis-benzimide) in parallel. The primary antibody is anti-transglutaminase K (NOVUS Biologicals, ref NB100-1844), and the secondary antibody is GAR-ELEXA 488 (Molecular Probes, ref A11008).

The fluorescence is measured by images (10 photos/well) using an INCell analyser™ 1000 (from GE Healthcare). The labelling is quantified by the measurement of fluorescence intensity normalised to the total number of cells (Integration of numerical data with the Developer Toolbox 1.5, GE Healthcare software).The procedure measures changes in amount of transglutaminase. The results obtained are presented below. Typically better results are obtained for examples having a higher percentage change in transglutaminase (TGK). The results obtained are:

| | CE1 | CE2 | CE3 | EX1 | EX2 | EX3 | EX4 | EX5 | EX6 | EX7 |
|---|---|---|---|---|---|---|---|---|---|---|
| TGK* | −1 | +12 | +36 | +64 | +85 | +152 | +176 | +152 | +59 | N.M. |

Footnote:
*is the percentage change in TGK.
N.M. indicates result not measured.

### Study 2

Example 8 (EX8): is an oil-in-water emulsion composition comprising approximately 50.3 wt % water, 4.1 wt % glycerine, 10 wt % dimethicone+dimethicone crosspolymer, 4.5 wt % hibiscus extract, 0.5 wt % hydrolysed rice protein, 3 wt % of Liposome soy isoflavone (commercially available from Mibelle), and 0.5 wt % of 3-ethyl ascorbic acid.

Example 9 (EX9): is an oil-in-water emulsion composition comprising approximately 50.8 wt % water, 4.1 wt % glycerine, 10 wt % dimethicone+dimethicone crosspolymer, 4.5 wt % hibiscus extract, 0.5 wt % hydrolysed rice protein, 2.5 wt % of Essenskin ceramide (commercially available from Sederma and is a mixture of homotaurine and calcium salt of hydroxymethionine), and 0.5 wt % of 3-ethyl ascorbic acid.

Each example is prepared by blending and mixing oil phase ingredients, and aqueous phase ingredients separately at a temperature of about 70° C. to ensure that all ingredients are solubilised in either water or oil. Once solubilised the oil phase and aqueous phase are blended at a temperature of about 70° C. until a homogenous emulsion composition is formed. Each example is then allowed to cool to ambient temperature.

### Testing

Invention Examples EX8 and EX9 are evaluated by an 8 week in vivo split face/neck/decollete double blinded randomised controlled design on women aged 45-60 years old presenting with mild to advanced signs of ageing. Each example is applied twice a day over the designated randomised half side of the face, neck and décolleté. Anti-ageing efficacy is evaluated by the clinical grading of numerous facial features including crows-feet wrinkles, firmness, evenness of skin tone, photodamage, peri-oral wrinkles and forehead wrinkles. Modified Griffiths' 10 point scales are used for each skin feature assessed where: 0=none (best possible skin condition), 1 to 3=mild, 4 to 6=moderate, and 7 to 9=severe (worst possible condition). Half point scores were assigned as necessary to accurately describe the skin feature. Typically better results are obtained for examples having a higher percentage grade change increase. The percentage grade changes have been normalised for changes in untreated skin. The mean % improvement in expert grading obtained for EX8-EX9 are reported as follows:

| | | | | Uneven |
|---|---|---|---|---|
| | | Photodamage | Firmness | Skin Tone |
| % Grade Change | EX8 | +15.3 | +16.1 | +17.3 |
| for Face | EX9 | +16.9 | +15.8 | +19.0 |
| % Grade Change | EX8 | +13.6 | +8.2 | +17.2 |
| for Neck | EX9 | +10.6 | +10.7 | +18.2 |
| % Grade Change | EX8 | +9.8 | +11.0 | +13.0 |
| for Décolleté | EX9 | +14.6 | +13.0 | +14.5 |

The performance results obtained indicate that the composition of the disclosed technology has improved firmness, reduces photodamage, and reduces uneven skin tone of face, neck and décolleté.

1. A method of decreasing or preventing signs of ageing of décolleté skin and optionally the face and/or neck skin comprising topically applying to the skin a composition comprising a cosmetically acceptable medium, a vitamin C, or derivative thereof, and a compound having two or more hydroxyl groups, wherein the compound has a molar mass of at least 150 g/mol and increases transglutaminase K expression in epidermal keratinocytes.

2. The method of claim 1, wherein the vitamin C, or derivative thereof is selected from the group consisting of ascorbic acid, O-substituted ascorbic acid or derivative thereof, sodium ascorbyl phosphate, ascorbyl glycoside, L-ascorbic acid, ascorbyl palmitate, retinyl ascorbate, tetrahexyldecyl ascorbate, magnesium ascorbyl phosphate, and mixtures thereof.

3. The method of claim 1, wherein the vitamin C or derivative thereof is present at 0.001 to 5 wt % of the composition.

4. The method of claim 1, wherein the compound having two or more hydroxyl groups has a molar mass of 150 to 2000 g /mol.

5. The method of claim 1, wherein the compound having two or more hydroxyl groups is provided by (i) a salt and/or derivative of hydroxymethionine, (ii) genestein, (iii) a Centella asiatica extract, (iv) a Cistus incanus extract, (v) a Polygonum bistorta extract or (vi) a Myrothamnus flabellifolia extract.

6. The method of claim 1, wherein the compound having two or more hydroxyl groups is ionic.

7. The method of claim 1, wherein the compound having two or more hydroxyl groups is non-ionic.

8. The method of claim 1, wherein the compound having two or more hydroxyl groups is aromatic.

9. The method of claim 8, wherein the compound having two or more hydroxyl groups is a polyphenol.

10. The method of claim 9, wherein the polyphenol is derived from a plant extract selected from the group consisting of (i) Centella asiatica, (ii) Cistus incanus, (iii) Polygonum bistorta, (iv) Myrothamnus flabellifolia, and mixtures thereof.

11. The method of claim 1, wherein the compound having two or more hydroxyl groups is non-aromatic.

12. The method of claim 11, wherein the compound having two or more hydroxyl groups is glyceryl glucoside.

13. The method of claim 11, wherein the compound having two or more hydroxyl groups is in the form of a mixture comprising a hydroxymethionine salt and homotaurine.

14. The method of claim 13, wherein the hydroxymethionine salt is a calcium salt of 2-hydroxymethionine.

15. The method of claim 1, wherein the compound having two or more hydroxyl groups is present in an amount ranging from 0.0001 wt % to 10 wt % of the composition.

16. The method of claim 1, wherein the composition is applied to the décolleté, and neck skin.

17. The method of claim 1, wherein the composition is applied to the décolleté, and face skin.

18. The method claim 1, wherein the composition is applied to the décolleté, face and neck skin.

19. The method of claim 1, wherein the compound having two or more hydroxyl groups comprises a calcium salt of hydroxymethionine.

20. The method of claim 1, wherein the compound having two or more hydroxyl groups is present in an amount ranging from 0.003 wt % to 3.5 wt % of the composition.
